(19) United States
(12) Patent Application Publication
(10) Pub. No.: US 2006/0013793 A1
(43) Pub. Date: Jan. 19, 2006

(54) Solid water-in-oil cosmetic emulsion

(76) Inventor: Agnes Themens, Bourg la Reine (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/182,849

(22) Filed: Jul. 18, 2005

Related U.S. Application Data
(60) Provisional application No. 60/601,990, filed on Aug. 17, 2004.
(60) Provisional application No. 60/602,328, filed on Aug. 18, 2004.

(30) Foreign Application Priority Data
Jul. 16, 2004 (FR) 04 51548
Jul. 16, 2004 (FR) 04 51550

(51) Int. Cl. A61K 8/892

(52) U.S. Cl. 424/70.12

(57) ABSTRACT

Disclosed herein is a solid water-in-oil emulsion comprising an aqueous phase emulsified in a fatty phase comprising at least one ester oil and at least one wax, and at least one silicone surfactant comprising at least one polyhydroxylated chain or at least one alkyl dimethicone copolyol chosen from C₈-C₂₂ alkyl dimethicone copolyols, as well as a process for making up or caring for skin, comprising applying to the skin the emulsion.

This application claims benefit of U.S. Provisional Application No. 60/601,990, filed Aug. 17, 2004, and U.S. Provisional Application No. 60/602,328, filed Aug. 18, 2004, the contents of which are incorporated herein by reference. This application also claims benefit of priority under 35 U.S.C. § 119 to French Patent Application No. 04 51548, filed Jul. 16, 2004, and French Patent Application No. 04 51550, filed Jul. 16, 2004, the contents of which are also incorporated by reference.

The present disclosure relates to a solid water-in-oil emulsion comprising at least one wax and at least one silicone surfactant, which may be used in the cosmetics field. The present disclosure also relates to a process for making up or caring for human skin, comprising applying the composition to the skin.

The makeup composition disclosed herein is, for example, a skin makeup composition, such as a foundation, an eyeshadow, a makeup rouge, a concealer product, a body makeup product or a lipstick. In one embodiment, the present disclosure relates to a foundation composition.

The care composition may be a skincare product such as a skincare base, a care cream (day cream, night cream or anti-wrinkle cream), a makeup base, a lipcare composition (lip balm), an antisun composition or self-tanning composition, and a deodorant.

Skin makeup products such as foundations are known in diverse galenical forms, such as loose powder, compact powder, cast solid product, stick, and fluid cream.

The cast solid products may be anhydrous or in the form of emulsions. These emulsions generally contain fatty substances such as oils and solid waxes, water and a particulate phase generally composed of fillers and pigments.

The solid emulsion does not flow under its own weight at room temperature and is suitable for packaging in a case: to apply the product, the user can take the product up directly by crumbling it using the fingers or an applicator such as a sponge. For good uptake of the product by the user, the solid emulsion should have a satisfactory hardness, such as a hardness that varies little over time, for example, after storage at temperatures above room temperature (25° C.). However, certain solid emulsions may show a drop in their hardness over time: the product then has a tendency to soften, making its uptake more difficult, or even unpleasant for the user, who may have difficulty in controlling the amount of product taken during the uptake. Furthermore, an initially solid product that becomes soft may not be appealing to the user.

Disclosed herein is a solid makeup or skincare composition that can have a stable hardness over time, for example, after storage for at least one month at 37° C.

Further disclosed herein is a solid composition that can have good hardness while at the same time having a rich, creamy texture when applied to the skin.

The present inventor has discovered that by using an ester oil, a wax and a particular emulsifying surfactant, it is possible to obtain a solid emulsion that has stable hardness over time, such as after one month at 37° C., and that also has a rich, creamy texture when applied to the skin.

Thus disclosed herein is a solid water-in-oil emulsion comprising an aqueous phase emulsified in a fatty phase comprising at least one ester oil and at least one wax, and at least one silicone surfactant comprising at least one polyhydroxylated chain.

Further disclosed herein is a solid water-in-oil emulsion comprising an aqueous phase emulsified with at least one emulsifying surfactant in a fatty phase comprising at least one ester oil and at least one wax, and at least one alkyl dimethicone copolyol chosen from C₈-C₂₂ alkyl dimethicone copolyols, wherein the emulsion has, after at least one month at 37° C., a hardness such that the penetration force is greater than or equal to 45 g.

Even further disclosed herein is a non-therapeutic cosmetic process for making up or caring for the skin, comprising applying to the skin the composition as defined above.

As used herein, the term “solid composition” means a composition that does not flow under its own weight at room temperature (25° C.) after one hour.

The solid emulsion disclosed herein comprises at least one ester oil.

The term “ester oil” means an oil that is liquid at room temperature (25° C.) comprising at least one ester functional group. The at least one ester oil used herein is chosen, for example, from monoesters.

The ester oil may, for example, be chosen from the monoesters of formula R₁COOR₂ wherein R₁ is chosen from linear and branched hydrocarbon-based chains comprising from 4 to 40 carbon atoms, such as from 4 to 30 carbon atoms and further such as from 7 to 20 carbon atoms, and R₂ is chosen from branched hydrocarbon-based chains comprising from 3 to 40 carbon atoms, such as from 10 to 30 carbon atoms and further such as from 16 to 26 carbon atoms.

Examples of the ester oils that may be mentioned include isodecyl neopentanoate; isocetyl octanoate; isononyl isononanoate, isodecyl isononanoate, tridecyl isononanoate; hexyl laurate, 2-hexyldecyl laurate; isopropyl myristate, isocetyl myristate, isotridecyl myristate, 2-octyldodecyl myristate; isopropyl palmitate, 2-ethylhexyl palmitate, isooctyl palmitate, isocetyl palmitate, isodecyl palmitate, isostearyl palmitate, 2-octyldecyl palmitate; isopropyl isostearate, 2-octyldodecyl stearate, isostearyl isostearate; 2-octyldodecyl erucate; and mixtures thereof.

The at least one ester oil may be present in the emulsion disclosed herein in an amount ranging, for example, from 5% to 20% by weight, such as from 7% to 15% by weight, and further such as from 8% to 12% by weight, relative to the total weight of the composition.

The fatty phase may also comprise at least one volatile silicone oil.

The term “volatile oil” means an oil (or non-aqueous medium) that can evaporate on contact with the skin in less than one hour, at room temperature and atmospheric pressure. The volatile oil is a volatile cosmetic oil, which is liquid at room temperature, for example, having a non-zero vapor pressure at room temperature and atmospheric pressure, such as having a vapor pressure ranging, for example, from 0.13 Pa to 40 000 Pa (10-3 to 300 mmHg), such as from 1.3 Pa to 13 000 Pa (0.01 to 100 mmHg), and further such as from 1.3 Pa to 1300 Pa (0.01 to 10 mmHg).

The volatile silicone oil may be chosen, for example, from linear and cyclic silicone oils with a viscosity at room temperature of less than 8 mm²/s comprising, for example, from 2 to 7 silicon atoms, wherein these silicone oils optionally comprise at least one group chosen from alkyl and alkoxy groups comprising from 1 to 10 carbon atoms. As the volatile silicone oils that may be used herein, mention may be made, for example, of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

The at least one volatile silicone oil may be present in an amount ranging from 5% to 40% by weight, such as from 10% to 30% by weight, and further such as from 15% to 30% by weight, relative to the total weight of the composition.

In at least one embodiment, the total content of water and the volatile silicone oil in the composition is less than or equal to 50% by weight, ranging, for example, from 30% to 50% by weight, such as from 40% to 50% by weight, relative to the total weight of the composition.

The fatty phase of the emulsion may comprise at least one additional oil other than the at least one ester oil and the at least one volatile silicone oil described above.

The at least one additional oil may be chosen, for example, from volatile and non-volatile hydrocarbon-based oils and fluoro oils.

The term “hydrocarbon-based oil” means an oil formed essentially from, or even consisting of, carbon and hydrogen atoms, and possibly oxygen and nitrogen atoms, and containing no silicon or fluorine atoms. It may comprise at least one group chosen from alcohol, ether, carboxylic acid, amine and amide groups.

The term “fluoro oil” means an oil comprising at least one fluorine atom.

As the additional volatile oil that may be used herein, mention may be made, for example, of:
hydrocarbon-based volatile oils comprising from 8 to 16 carbon atoms, and mixtures thereof, for example, branched C₈-C₁₆ alkanes, such as C₈-C₁₆ isoalkanes (also known as isoparaffins), isododecane, isodecane and isohexadecane, such as the oils sold under the trade names “Isopar” and “Permethyl”, branched C₈-C₁₆ esters, such as isohexyl neopentanoate and mixtures thereof; in one embodiment, isododecane is used;
volatile fluoro oils such as nonafluoroethoxybutane, nonafluoromethoxybutane, decafluoropentane, tetradecafluorohexane and dodecafluoropentane, and mixtures thereof,
and mixtures thereof.

As additional non-volatile oil that may be used herein, mention may be made, for example, of hydrocarbon-based oils of mineral or synthetic origin, such as linear or branched hydrocarbons, for instance liquid paraffin or derivatives thereof, liquid petroleum jelly, polydecenes, hydrogenated polyisobutene such as PARLEAM sold by the company Nippon Oil Fats, and squalane of synthetic or plant origin.

Additional non-volatile oils that may also be mentioned include, for example, hydrocarbon-based oils of plant origin with a high triglyceride content, comprising fatty acid esters of glycerol, the fatty acids of which may have varied chain lengths, wherein these chains may be linear or branched, and saturated or unsaturated, for example, triglycerides of a fatty acid comprising, for example, from 4 to 22 carbon atoms, such as heptanoic or octanoic acid triglycerides, and capric/caprylic acid triglycerides, or alternatively hydroxylated triglycerides, such as sweet almond oil, beauty-leaf oil, palm oil, grapeseed oil, sesame seed oil, arara oil, rapeseed oil, sunflower oil, cottonseed oil, apricot oil, castor oil, alfalfa oil, marrow oil, blackcurrant oil, macadamia oil, musk rose oil, hazelnut oil, avocado oil, jojoba oil, olive oil, cereal (corn, wheat, barley or rye) germ oil and shea butter oil;

C₈-C₂₆ higher fatty acids such as oleic acid, linoleic acid, linolenic acid or isostearic acid; C₈-C₂₆ higher fatty alcohols such as oleyl alcohol, linoleyl alcohol, linolenyl alcohol, isostearyl alcohol or octyldodecanol; synthetic ethers comprising at least 7 carbon atoms, silicone oils such as linear, optionally phenylated polydimethylsiloxanes (PDMS) that are liquid at room temperature, such as phenyl trimethicones, phenyl trimethylsiloxy diphenylsiloxanes, diphenyl dimethicones, diphenyl methyldiphenyl trisiloxanes and 2-phenylethyl trimethylsiloxysilicates, which are liquid, optionally substituted with at least one group chosen from aliphatic and aromatic groups, such as alkyl, alkoxy or phenyl groups, which are pendent and/or at the end of a silicone chain, wherein these groups comprise from 2 to 24 carbon atoms and are optionally fluorinated, or comprise at least one functional group chosen, for example, from hydroxyl, thiol and amine groups;
and mixtures thereof.

For example, the at least one additional oil is present in an amount ranging from 0.1% to 10% by weight, such as from 0.1% to 5% by weight, relative to the total weight of the composition. In one embodiment of the emulsion disclosed herein, the content of the at least one additional oil is 0% by weight relative to the total weight of the composition.

In at least one embodiment, the emulsion comprises as sole oil an ester oil and optionally a volatile silicone oil, as described above.

In at least one embodiment, the at least one wax present in the emulsion disclosed herein is chosen from waxes with a hardness ranging from 5 MPa to 9 MPa, such as from 6 MPa to 9 MPa, and further such as from 7 MPa to 9 MPa.

As used herein, the term “wax” means a lipophilic fatty compound that is solid at room temperature (25° C.), with a reversible solid/liquid change of state, having a melting point of greater than 30° C. which may be up to 200° C., a hardness of greater than 0.5 MPa, and having an anisotropic crystal organization in the solid state. By bringing the wax to its melting point, it is possible to make it miscible with oils and to form a microscopically homogeneous mixture, but on returning the temperature of the mixture to room temperature, recrystallization of the wax in the oils of the mixture is obtained.

The hardness of the wax is determined by measuring the compression force, measured at 20° C. using the texturometer sold under the name TA-XT2i by the company Rheo, equipped with a stainless-steel cylinder 2 mm in diameter travelling at a measuring speed of 0.1 mm/s, and penetrating into the wax to a penetration depth of 0.3 mm. To perform the hardness measurement, the wax is melted at a temperature equal to the melting point of the wax+20° C. The molten wax is poured into a container 30 mm in diameter and 20 mm deep. The wax is recrystallized at room temperature (25° C.) for 24 hours and is then stored for at least 1 hour at 20° C. before performing the hardness measurement. The hardness value is the measured compression force divided by the surface area of the texturometer cylinder in contact with the wax.

As disclosed herein, the waxes may be chosen from hydrocarbon-based waxes, silicone waxes and fluoro waxes, optionally comprising at least one functional group chosen from ester and hydroxyl functional groups.

The wax having the hardness as defined above may be chosen from carnauba wax, microcrystalline waxes, ozokerites, hydrogenated jojoba oil, polyethylene waxes such as the wax sold under the name “Performalene 400 Polyethylene” by the company New Phase Technologies, silicone waxes, for instance poly(C24-C28)alkylmethyldimethylsiloxane, such as the product sold under the name “Abil Wax 9810” by the company Goldschmidt, palm butter, the C20-C40 alkyl stearate sold under the name “Kester Wax K82H” by the company Kester Keunen, stearyl benzoate, shellac wax, and mixtures thereof. For example, a wax chosen from carnauba wax, candelilla wax, ozokerites, hydrogenated jojoba oil and polyethylene waxes is used. In at least one embodiment, the wax is chosen from candelilla wax and ozokerite, and mixtures thereof.

The at least one wax may be present in the composition disclosed herein in an amount ranging from 1% to 10% by weight, such as from 2% to 7% by weight, and further such as from 2% to 5% by weight, relative to the total weight of the composition.

The emulsion as disclosed herein may comprise at least one silicone surfactant comprising at least one polyhydroxylated chain, which may be chosen from silicone polymers comprising at least one polyhydroxylated chain and silicone elastomers comprising at least one polyhydroxylated chain. The term “polyhydroxylated chain” means a hydrocarbon-based chain comprising at least two hydroxyl groups.

The silicone polymers of the formula (I′) below may, for example, be used:
R¹ₐR²_{b}R³_{c}SiO_{(4-a-b-c)/2} (I′)
wherein:
a) a ranges from 1 to 2.5; and b and c, which may be identical or different, range from 0.001 to 1.5,
b) R¹, which may be identical or different, is chosen from:
   C₁ to C₃₀ alkyl radicals, where appropriate substituted with at least one entity chosen from fluorine atoms and amino and carboxyl groups,
   aryl and aralkyl radicals, and
   the radicals of formula (II):
      —C_{d}H_{2d}—O—(C₂H₄O)ₑ(C₃H₆O)_{f}R⁴ (II)
wherein:
   R⁴ is chosen from C₄ to C₃₀ hydrocarbon-based radicals and a radical R⁵—(CO)— wherein R⁵ is chosen from C₁ to C₃₀ hydrocarbon-based radicals, and
   d, e, and f are integers such that d ranges from 0 to 15, and e and f, which may be identical or different, range from 0 to 50, and
   combinations thereof,
c) R², which may be identical or different, is a radical of formula (III) below:
   -Q-O—X (III)
wherein:
   Q is a divalent C₂ to C₂₀ hydrocarbon-based radical which may comprise at least one bond chosen from ether bonds and ester bonds, and
   X is a polyhydroxylated hydrocarbon-based radical,
d) R³, which may be identical or different, is an organosiloxane group of formula (IV): 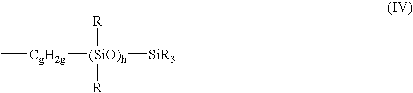
wherein:
   the radicals R, which may be identical or different, are each a radical chosen from C₁ to C₃₀ alkyl radicals, where appropriate substituted with at least one fluorine atom, and aryl and aralkyl radicals,
   g and h are integers such that g ranges from 1 to 5 and h ranges from 0 to 500.

When the radicals R are each a radical chosen from C₁ to C₃₀ alkyl radicals, where appropriate substituted with at least one fluorine atom, aryl radicals and aralkyl radicals, they have the same meaning as the radical R¹ as defined above.

It should be noted that the radicals R¹, R² and R³ of the silicone polymers of formula (I′), as defined above, are not sequentially distributed, but are randomly distributed, i.e., they appear in the structure of the polymer without any determined order.

In a):
a ranges, for example, from 1.2 to 2.3 and b and c, which may be identical or different, range, for example, from 0.05 to 1.

In b):
when R¹ is an alkyl radical, it may be a C₁ to C₃₀ alkyl radical, for example, a C₁ to C₂₅ alkyl radical, such as a C₁ to C₂₀ alkyl radical, further such as a C, to C₁₀ alkyl radical and even further such as a C₁ to C₆ alkyl radical. In one embodiment, R¹ is a C₁ to C₄ alkyl radical. For example, R¹ may be chosen from methyl, ethyl, n-propyl, isopropyl, n-butyl, isobutyl, tert-butyl, pentyl, hexyl, heptyl, octyl, nonyl, decyl, undecyl and lauryl radicals. It may also be a cycloalkyl radical such as a cyclopropyl, a cyclobutyl, a cyclopentyl or a cyclohexyl. It may also be a linear or branched monounsaturated or polyunsaturated alkyl radical. It may also be an alkyl radical substituted with at least one fluorine atom, such as trifluoropropyl or heptadecafluorodecyl. It may also be an alkyl radical substituted with at least one amino group, such as 2-aminoethyl, 3-aminopropyl or 3-(2-aminoethyl)aminopropyl. It may also be an alkyl group substituted with at least one carboxyl group, such as 3-carboxypropyl;
R¹ may also be an aryl or aralkyl radical such as a phenyl radical, a tolyl radical, a benzyl radical or a phenethyl radical;
R¹ may also be an organic group of formula (II):
   —C_{d}H_{2d}—O—(C₂H₄O)ₑ(C₃H₆O)_{f}R⁴ (II)

In one embodiment, R¹ may be a hydroxylated radical or a radical derived from the addition reaction of a saturated or unsaturated, linear or branched alkenyl ether, in which d=0 and thus of formula:
—O—(C₂H₄O)ₑ(C₃H₆O)_{f}R⁴

In this case, when e and f are equal to zero, then R¹ is an alkoxy group comprising from 4 to 30 carbon atoms, for example, a C₄ to C₁₀ lower alkoxy radical, such as butoxy or pentoxy, or a C₁₁ to C₃₀ higher alkoxy radical, such as oleoxy or stearoxy, for example, cetyl alcohol, oleyl alcohol and stearyl alcohol, or a radical derived from an acid or from a fatty acid, such as acetic acid, lactic acid, butyric acid, oleic acid, stearic acid or behenic acid.

When e and f are greater than 1, then R¹ is a hydroxyl radical derived from the addition reaction of an alkylene oxide.

When e and f are equal to zero, d is, for example, equal to 3, 5 or 11. In this case, R¹, depending on the nature of the substituent R⁴, is an allyl ether, pentenyl ether or undecenyl ether radical, or an allyl stearyl ether, pentenyl behenyl ether or undecenyl oleyl ether radical.

When e and f are other than zero, an alkoxy radical and an ester radical are present via a polyoxyalkylene group.

Irrespective of the nature of e and f, d ranges, for example, from 3 to 5.

In one embodiment, the radical R¹ may be any one of the radicals defined above, or a combination of two or more of these radicals.

For example, R¹ is an alkyl radical chosen from a methyl radical and a lauryl radical, and combinations thereof.

Moreover, when R¹ corresponds to two or more radicals, for example, a methyl radical and a lauryl radical, these radicals appear randomly in the structure, and with a frequency that may be different for each of the radicals.

For example, at least 50% of the radicals R¹, such as at least 70% of the radicals R¹ and further such as 100% of the radicals R¹, are methyl radicals.

In c):
Q may, for example, be a divalent hydrocarbon-based radical chosen from:
—(CH₂)₂—, —(CH₂)₃—, —CH₂CH(CH₃)—CH₂, —(CH₂)₄—, —(CH₂)₅—, —(CH₂)₆—, —(CH₂)₇—, —(CH₂)₈—, —(CH₂)₂—CH(CH₂CH₂—CH₃)—, —CH₂—CH(CH₂CH₃)—, —(CH₂)₃—O—(CH₂)₂—, —(CH₂)₃—O—(CH₂)₂—O—(CH₂)₂—, —(CH₂)₃—O—CH₂CH(CH₃)— and —CH₂—CH(CH₃)—COO(CH₂)₂—.

In at least one embodiment, Q is a divalent radical chosen from —(CH₂)₂— and —(CH₂)₃—.
X may, for example, be a polyhydroxylated hydrocarbon-based radical comprising at least two hydroxyl residues, for example, a hydrocarbon-based group chosen from glyceryl derivatives and saccharide derivatives.

The glycerol residues may be compounds of the following formulae, wherein Q has the same meaning as in formula (III) above, and s and t are integers ranging from 1 to 20, such as from 1 to 15, further such as from 1 to 10 and even further such as from 1 to 5.

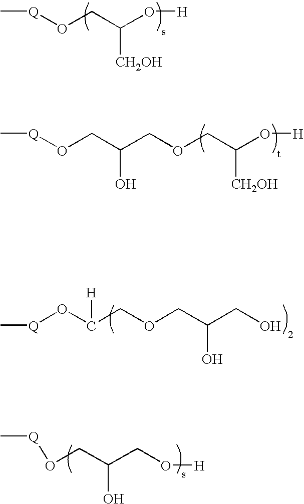

In the above formulae, at least one hydroxyl group may be replaced with at least one group chosen from alkoxy groups and ester groups.

The saccharide radicals that may be used in the general formula (III) may be of monosaccharide type, such as glycosyl, mannosyl, galactosyl, ribosyl, arabinosyl, xylosyl or fructosyl groups, of oligosaccharide type, such as maltosyl, cellobiosyl, lactosyl or maltotriosyl, or of polysaccharide type, such as cellulose or starch.

For example, the saccharide groups are of monosaccharide or oligosaccharide type.

In d):
the radicals R, which may be identical or different, may each be, for example, a radical chosen from C₁ to C₂₀, such as from C₁ to C₁₀ and further such as from C₁ to C₆ alkyl radicals, where appropriate substituted with at least one fluorine atom. When the radicals R are each a radical chosen from the alkyl radicals as defined above, where appropriate substituted with at least one fluorine atom, they have the same meaning as the radical R¹ as defined above.
in at least one embodiment, g is equal to 2.
in at least one embodiment, h ranges from 1 to 50.

For example, in at least one embodiment, the silicone polymer of general formula (I′), which may be used in the cosmetic compositions disclosed herein, is such that:
a ranges from 1 to 1.4, and b and c, which may be identical or different, range from 0.02 to 0.03, and
R¹ is a C₁ to C₁₀, such as C₁ to C₆ and further such as C₁ to C₄, alkyl radical,
R² is a radical of formula (IIIA):
   —C₃H₆O[CH₂CH(OH)CH₂O]ₙH (IIIA)
   wherein n ranges from 1 to 5, and
R³ is a radical of the formula (IVA):
   —C₂H₄(CH₃)₂SiO[(CH₃)₂SiO]ₘSi(CH₃)₃ (IVA)
   wherein m ranges from 3 to 9.

In at least one embodiment, the silicone polymer of formula (I′), which may be used in the cosmetic compositions disclosed herein, is such that:
a ranges from 1 to 1.4, and b and c, which may be identical or different, range from 0.02 to 0.04,
R¹ is a methyl radical,
R² is a radical of the formula (IIIA) wherein n ranges from 1 to 5, and
R³ is a radical of the formula (IVA) wherein m ranges from 3 to 9.

For example, the silicone polymer of formula (I′) used in the composition disclosed herein is chosen from polyglyceryl-3 polymethylsiloxyethyl dimethicone, laurylpolyglyceryl-3 polymethylsiloxyethyl dimethicone and polyglyceryl-3 disiloxane dimethicone, the respective formulae of which are:
Polyglyceryl-3 polymethylsiloxyethyl dimethicone (formula (V)): 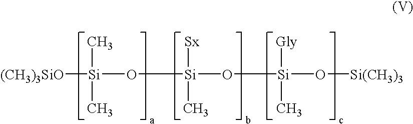
   wherein:
Sx: —C₂H₄[(CH₃)₂SiO]ₘSi(CH₃)₃
Gly: —C₃H₆O[CH₂—CH(OH)CH₂O]ₙH
a ranges from 1 to 1.4,
b and c independently range from 0.02 to 0.04,
m ranges from 3 to 9, and
n ranges from 1 to 5.
   Laurylpolyglyceryl-3 polymethylsiloxyethyl dimethicone (formula (VI)): 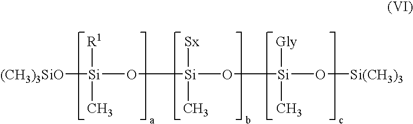
      wherein Sx, Gly, a, b, c, m and n have the same meaning as above and R¹ is either a methyl radical or a lauryl radical;
   Polyglyceryl-3 disiloxane dimethicone (formula (VII)): 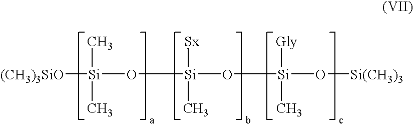
      wherein Gly, a, b, c, m and n have the same meaning as above, and
      Sx: —O(CH₃)₂SiO—Si(CH₃)₃.

The silicone polymers of formula (I′) are described in detail in European patent application EP 1 213 316, which is incorporated herein by reference.

In one embodiment, the silicone polymer of general formula (I′) is chosen from the polymers sold by the company Shin-Etsu under the references KF 6100, KF 6104 and KF 6105.

The polymer sold under the reference KF 6104 is, for example, suitable for preparing the cosmetic compositions as disclosed herein.

An emulsifying elastomeric crosslinked organopolysiloxane comprising at least one polyhydroxylated chain may also be used as emulsifier.

The elastomeric crosslinked organopolysiloxane may be obtained by a crosslinking addition reaction of diorganopolysiloxane comprising at least one hydrogen linked to silicon and of polyhydroxylated or polyoxyalkylenated compounds comprising at least one ethylenically unsaturated group, such as in the presence of a platinum catalyst.

For example, the elastomeric crosslinked organopolysiloxane is obtained by a crosslinking addition reaction of (A) diorganopolysiloxane comprising at least two hydrogens each linked to a silicon, and (B) glycerolated compounds comprising at least two ethylenically unsaturated groups, such as in the presence of (C) a platinum catalyst.

Further, for example, the organopolysiloxane may be obtained by reaction of a polyglycerolated compound comprising dimethylvinylsiloxy end groups and methylhydrogenopolysiloxane comprising trimethylsiloxy end groups, in the presence of a platinum catalyst.

Compound (A) is the base reagent for the formation of elastomeric organopolysiloxane and the crosslinking is performed by an addition reaction of compound (A) with compound (B) in the presence of the catalyst (C).

Compound (A) is, for example, an organopolysiloxane comprising at least two hydrogen atoms linked to different silicon atoms in each molecule.

Compound (A) may have any molecular structure, such as a linear chain or branched chain structure or a cyclic structure.

Compound (A) may have a viscosity at 25° C. ranging from 1 to 50 000 centistokes, for example, in order to have good miscibility with compound (B).

The organic groups linked to silicon atoms of the compound (A) may be chosen from alkyl groups comprising from 1 to 18 carbon atoms, such as methyl, ethyl, propyl, butyl, octyl, decyl, dodecyl (or lauryl), myristyl, cetyl or stearyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group. The organic group is, for example, chosen from methyl, phenyl and lauryl groups.

Compound (A) may thus be chosen from methylhydrogenopolysiloxanes comprising trimethylsiloxy end groups, dimethylsiloxane-methylhydrogenosiloxane copolymers comprising trimethylsiloxy end groups, dimethylsiloxane-methylhydrogenosiloxane cyclic copolymers, or dimethylsiloxane-methylhydrogenosiloxane-laurylmethylsiloxane copolymers comprising trimethylsiloxy end groups.

Compound (B) may be a polyglycerolated compound of formula (B′) below:
CₘH₂ₘ₋₁—O—[Gly]n-CₘH₂ₘ₋₁ (B′)
wherein m is an integer ranging from 2 to 6, n is an integer ranging from 2 to 200, such as from 2 to 100, further such as from 2 to 50; for example, n ranges from 2 to 20, such as from 2 to 10 and further such as from 2 to 5; in one embodiment, n equals to 3; Gly is: —CH₂—CH(OH)—CH₂—O— or —CH₂—CH(CH₂OH)—O—.

For example, the sum of the number of ethylenic groups per molecule of compound (B) and the number of hydrogen atoms linked to silicon atoms per molecule of compound (A) is at least 4.

Compound (A) is, for example, present in an amount such that the molar ratio between the total amount of hydrogen atoms linked to silicon atoms in compound (A) and the total amount of all the ethylenically unsaturated groups in compound (B) ranges from 1:1 to 20:1.

Compound (C) is the crosslinking reaction catalyst, and is, for example, chloroplatinic acid, chloroplatinic acid-olefin complexes, chloroplatinic acid-alkenylsiloxane complexes, chloroplatinic acid-diketone complexes, platinum black and platinum on a support.

The catalyst (C) is, for example, present in an amount ranging from 0.1 to 1000 parts by weight, such as from 1 to 100 parts by weight, as clean platinum metal per 1000 parts by weight of the total amount of compounds (A) and (B).

The emulsifying silicone elastomer may be conveyed in gel form in at least one of the hydrocarbon-based oil and the silicone oil. In these gels, the emulsifying elastomer is often in the form of non-spherical particles.

Polyglycerolated elastomers are described, for example, in patent application WO-A-2004/024 798, the content of which is incorporated herein by reference.

Polyglycerolated silicone elastomers that may be used include, for example, those sold under the names “KSG-710”, “KSG-810”, “KSG-820”, “KSG-830” and “KSG-840” by the company Shin-Etsu.

The silicone surfactant comprising at least one polyhydroxylated chain may be present in the emulsion in an amount ranging from 0.1% to 5% by weight, such as from 1% to 3% by weight, relative to the total weight of the composition.

The emulsion disclosed herein may comprise as emulsifier at least one alkyl dimethicone copolyol chosen from C₈-C₂₂ alkyl dimethicone copolyols, i.e., an oxypropylenated and/or oxyethylenated polymethyl (C₈-C₂₂)alkyl dimethyl methyl siloxane.

The C₈-C₂₂ alkyl dimethicone copolyol is, for example, a compound of formula (I) below: 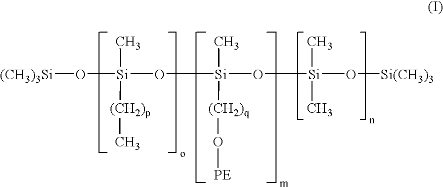
wherein:
PE is (—C₂H₄O)ₓ—(C₃H₆O)_{y}—R, wherein R is chosen from a hydrogen atom and alkyl radicals comprising from 1 to 4 carbon atoms, x ranges from 0 to 100 and y ranges from 0 to 80, x and y are not simultaneously 0,
m ranges from 1 to 40,
n ranges from 10 to 200,
o ranges from 1 to 100,
p ranges from 7 to 21, and
q ranges from 0 to 4;
   for example, in one embodiment:
R═H,
m=1 to 10,
n=10 to 100,
o=1 to 30,
p=15, and
q=3.

The C₈-C₂₂ alkyl dimethicone copolyols that may be mentioned include, for example, cetyl dimethicone copolyol, such as the product sold under the name Abil EM-90 by the company Goldschmidt.

The C₈-C₂₂ alkyl dimethicone copolyol may be present in the emulsion in an amount ranging, for example, from 0.1% to 5% by weight, such as from 1% to 3% by weight, relative to the total weight of the composition.

The emulsion as disclosed herein may comprise at least one additional emulsifying surfactant, which is independently different from the at least one silicone surfactant comprising at least one polyhydroxylated chain or from the C₈-C₂₂ alkyl dimethicone copolyols described above. The at least one additional emulsifying surfactant is chosen, for example, from surfactants that allow a water-in-oil emulsion to be obtained, such as a surfactant with an HLB (hydrophilic/lipophilic balance) of less than 7; such an emulsifying surfactant may be chosen, for example, from fatty acid esters of polyols, for instance glyceryl or sorbitol mono-, di-, tri- or sesqui-oleates or -stearates, glyceryl or polyethylene glycol laurates; alkyl or alkoxy dimethicone copolyols comprising an alkyl or alkoxy chain that is pendent or at the end of the silicone skeleton, for example, comprising from 6 to 22 carbon atoms; polymers such as polyoxyalkylenated fatty acid esters of glycol, and silicone surfactants comprising at least one polyhydroxylated chain.

As the at least one additional surfactant that may be used herein to obtain a W/O emulsion, mention may be made, for example, of polymers such as polyoxyalkylenated fatty acid esters of glycol with water-in-oil emulsifying properties.

The fatty acid ester of the emulsifying polymer is, for example, polyhydroxylated. In one embodiment, this polymer is a block polymer, such as of ABA structure, comprising poly(hydroxylated ester) blocks and polyethylene glycol blocks.

The fatty acid ester of the emulsifying polymer defined above generally has a chain comprising from 12 to 20 carbon atoms such as from 14 to 18 carbon atoms. The esters may be chosen, for example, from oleates, palmitates and stearates.

The polyethylene glycol blocks of the emulsifying polymer as defined above comprise, for example, from 4 to 50 mol of ethylene oxide such as from 20 to 40 mol of ethylene oxide.

One polymer surfactant that is suitable for producing the compositions disclosed herein is, for example, 30 EO polyethylene glycol dipolyhydroxystearate, sold under the trade name “Arlacel P 135” by the company ICI.

The at least one additional surfactant other than the silicone surfactant comprising at least one polyhydroxylated chain or the C₈-C₂₂ alkyl dimethicone copolyol described above may be present in the emulsion disclosed herein in an amount ranging, for example, from 0.05% to 3% by weight, such as from 0.1% to 2% by weight, relative to the total weight of the composition.

The aqueous phase of the solid emulsion disclosed herein comprises water. The water may be chosen from floral water such as cornflower water, mineral water such as eau de Vittel, eau de Lucas or eau de La Roche Posay, and spring water. The water may be present in the emulsion disclosed herein in an amount ranging, for example, from 10% to 40% by weight, such as from 15% to 30% by weight and further such as from 15% to 25% by weight, relative to the total weight of the composition.

The aqueous phase may also comprise solvents that are water-miscible (at room temperature of 25° C.), for instance monoalcohols comprising from 2 to 6 carbon atoms, such as ethanol or isopropanol;
polyols such as those comprising from 2 to 20 carbon atoms, further such as those comprising from 2 to 10 carbon atoms and even further such as those comprising from 2 to 6 carbon atoms, for example, glycerol, propylene glycol, butylene glycol, pentylene glycol, hexylene glycol, dipropylene glycol or diethylene glycol;
glycol ethers (for example, those comprising from 3 to 16 carbon atoms) such as mono-, di- or tripropylene glycol (C₁-C₄)alkyl ethers or mono-, di- or triethylene glycol (C₁-C₄)alkyl ethers;
and mixtures thereof.

The emulsion disclosed herein may comprise at least one water-miscible organic solvent, such as a polyol, in an amount ranging from 1% to 20% by weight, such as from 3% to 15% by weight, relative to the total weight of the composition.

The aqueous phase may also comprise at least one stabilizer chosen, for example, from sodium chloride, magnesium dichloride and magnesium sulfate.

The aqueous phase may also comprise any water-soluble or water-dispersible compound that is compatible with an aqueous phase, such as gelling agents, film-forming polymers, thickeners and surfactants, and mixtures thereof.

The aqueous phase may be present in the emulsion disclosed herein in an amount ranging, for example, from 25% to 50% by weight, such as from 25% to 45% by weight, and further such as from 25% to 35% by weight, relative to the total weight of the emulsion.

The fatty phase of the emulsion may also comprise at least one pasty fatty substance.

As used herein, the term “pasty” means a lipophilic fatty compound which undergoes a reversible solid/liquid change of state, having in the solid state an anisotropic crystal organization, and comprising at a temperature of 23° C. a liquid fraction and a solid fraction.

As used herein, the term “pasty compound” means a compound having a hardness at 20° C. ranging from 0.001 to 0.5 MPa such as from 0.002 to 0.4 MPa.

The hardness of the pasty compound is measured according to a method of penetration of a probe in a sample of the compound, for example, using a texture analyzer (for example the TA-XT2i machine from Rheo) equipped with a stainless-steel cylinder 2 mm in diameter. The hardness measurement is performed at 20° C. at the center of five samples. The cylinder is introduced into each sample at a pre-speed of 1 mm/s and then at a measuring speed of 0.1 mm/s, the penetration depth being 0.3 mm. The hardness value revealed is that of the maximum peak.

This pasty compound is also, at a temperature of 23° C., in the form of a liquid fraction and a solid fraction. In other words, the starting melting point of the pasty compound is less than 23° C. The liquid fraction of the pasty compound measured at 23° C. may be present in an amount ranging from 9% to 97% by weight of the compound. This liquid fraction at 23° C. is present, for example, in an amount ranging from 15% to 85% by weight, such as from 40% to 85% by weight, relative to the weight of the compound.

The liquid fraction by weight of the pasty compound at 23° C. is equal to the ratio of the heat of fusion consumed at 23° C. to the heat of fusion of the pasty compound.

The heat of fusion of the pasty compound is the heat consumed by the compound to change from the solid state to the liquid state. The pasty compound is said to be in the solid state when all of its mass is in solid crystalline form. The pasty compound is said to be in the liquid state when all of its mass is in liquid form.

The heat of fusion of the pasty compound is equal to the area under the curve of the thermogram obtained using a differential scanning calorimeter (DSC), such as the calorimeter sold under the name MDSC 2920 by the company TA Instrument, with a temperature rise of 5 or 10° C. per minute, according to standard ISO 11357-3:1999. The heat of fusion of the pasty compound is the amount of energy required to make the compound change from the solid state to the liquid state. It is expressed in J/g.

The heat of fusion consumed at 23° C. is the amount of energy absorbed by the sample to change from the solid state to the state that it has at 23° C., comprising a liquid fraction and a solid fraction.

The liquid fraction of the pasty compound, measured at 32° C., is present in an amount ranging, for example, from 30% to 100% by weight, such as from 80% to 100% by weight, and further such as from 90% to 100% by weight, relative to the weight of the compound. When the liquid fraction of the pasty compound measured at 32° C. is equal to 100%, the temperature of the end of the melting range of the pasty compound is less than, or equal to 32° C.

The liquid fraction of the pasty compound measured at 32° C. is equal to the ratio of the heat of fusion consumed at 32° C. to the heat of fusion of the pasty compound. The heat of fusion consumed at 32° C. is calculated in the same manner as the heat of fusion consumed at 23° C.

The pasty compound may, for example, be chosen from synthetic compounds and compounds of plant origin. A pasty compound may be obtained by synthesis from starting materials of plant origin.

The pasty compound may, for example, be chosen from:
polymer or non-polymer silicone compounds,
polymer or non-polymer fluoro compounds,
vinyl polymers, such as:
   olefin homopolymers,
   olefin copolymers,
   hydrogenated diene homopolymers and copolymers,
   linear or branched oligomers, which are homopolymers or copolymers of alkyl (meth)acrylates comprising, for example, at least one alkyl group chosen from C₈-C₃₀ alkyl groups,
   oligomers, which are homopolymers and copolymers of vinyl esters comprising at least one alkyl group chosen from C₈-C₃₀ alkyl groups,
   oligomers, which are homopolymers and copolymers of vinyl ethers comprising at least one alkyl group chosen from C₈-C₃₀ alkyl groups,
liposoluble polyethers resulting from the polyetherification between one or more C₂-C₁₀₀, such as C₂-C₅₀, diols,
esters,
and mixtures thereof.

The pasty compound is, for example, chosen from polymers, such as hydrocarbon-based polymers.

Silicone and/or Fluoro Pasty Compounds

In one embodiment, the silicone and fluoro pasty compound is polymethyl trifluoropropyl methylalkyl dimethylsiloxane, sold under the name X22-1088 by Shin-Etsu.

Polyether Pasty Compounds

Among the liposoluble polyethers, examples include copolymers of ethylene oxide and/or of propylene oxide with C₆-C₃₀ long-chain alkylene oxides, for example, such that the weight ratio of the ethylene oxide and/or of the propylene oxide to the alkylene oxides in the copolymer ranges from 5:95 to 70:30. In this family, mention will be made, for example, of copolymers such that the long-chain alkylene oxides are arranged in blocks with an average molecular weight ranging from 1000 to 10 000, for example, a polyoxyethylene/polydodecyl glycol block copolymer such as the ethers of dodecanediol (22 mol) and polyethylene glycol (45 EO) sold under the brand name Elfacos ST9 by Akzo Nobel.

Among the esters, examples include:
esters of a glycerol oligomer, such as diglycerol esters, for example, condensates of adipic acid and glycerol, for which some of the hydroxyl groups of the glycerols have reacted with a mixture of fatty acids such as stearic acid, capric acid, stearic acid and isostearic acid and 12-hydroxystearic acid, for instance those sold under the brand name Softisan 649 by the company Sasol,
arachidyl propionate sold under the brand name Waxenol 801 by Akzo,
phytosterol esters,
non-crosslinked polyesters resulting from polycondensation between a linear or branched C₄-C₅₀ dicarboxylic acid or polycarboxylic acid and a C₂-C₅₀ diol or polyol,
aliphatic esters of an ester resulting from the esterification of an aliphatic hydroxycarboxylic acid ester with an aliphatic carboxylic acid; and mixtures thereof.

The aliphatic carboxylic acid comprises from 4 to 30 such as from 8 to 30 carbon atoms. It is chosen, for example, from hexanoic acid, heptanoic acid, octanoic acid, 2-ethylhexanoic acid, nonanoic acid, decanoic acid, undecanoic acid, dodecanoic acid, tridecanoic acid, tetradecanoic acid, pentadecanoic acid, hexadecanoic acid, hexyldecanoic acid, heptadecanoic acid, octadecanoic acid, isostearic acid, nonadecanoic acid, eicosanoic acid, isoarachidic acid, octyldodecanoic acid, heneicosanoic acid and docosanoic acid, and mixtures thereof.

The aliphatic carboxylic acid is, for example, branched.

The aliphatic hydroxycarboxylic acid ester is, for example, derived from a hydroxylated aliphatic carboxylic acid comprising from 2 to 40 carbon atoms, such as from 10 to 34 carbon atoms and further such as from 12 to 28 carbon atoms, and from 1 to 20 hydroxyl groups, such as from 1 to 10 hydroxyl groups and further such as from 1 to 6 hydroxyl groups. The aliphatic hydroxycarboxylic acid ester is chosen, for example, from:
a) partial or total esters of saturated linear monohydroxylated aliphatic monocarboxylic acids;
b) partial or total esters of unsaturated monohydroxylated aliphatic monocarboxylic acids;
c) partial or total esters of saturated monohydroxylated aliphatic polycarboxylic acids;
d) partial or total esters of saturated polyhydroxylated aliphatic polycarboxylic acids;
e) partial or total esters of C₂ to C₁₆ aliphatic polyols that have reacted with a monohydroxylated or polyhydroxylated aliphatic monocarboxylic or polycarboxylic acid,
and mixtures thereof.

The aliphatic esters of an ester are, for example, chosen from:
the ester resulting from the esterification reaction of hydrogenated castor oil with isostearic acid in proportions of 1 to 1 (1/1) or hydrogenated castor oil monoisostearate, the ester resulting from the esterification reaction of hydrogenated castor oil with isostearic acid in proportions of 1 to 2 (1/2) or hydrogenated castor oil diisostearate,
the ester resulting from the esterification reaction of hydrogenated castor oil with isotearic acid in proportions of 1 to 3 (1/3) or hydrogenated castor oil triisostearate,
and mixtures thereof.

Among the pasty compounds of plant origin, examples include an oxyethylenated (5 EO) oxypropylenated (5 PO) mixture of soybean sterols and of pentaerythritol, sold under the name LANOLIDE by the company Vevy.

A pasty fatty substance that is used herein is, for example, a block copolymer of ethylene oxide and/or propylene oxide and of an alkylene oxide comprising from 6 to 40 carbon atoms, wherein the copolymer has a weight-average molecular weight ranging from 5000 to 8000.

For example, the alkylene oxide of the copolymer may comprise from 6 to 30 carbon atoms, such as from 8 to 20 carbon atoms, further such as from 10 to 18 carbon atoms and even further such as from 10 to 14 carbon atoms.

The weight-average molecular weight of the copolymer ranges, for example, from 5000 to 8000, such as from 5500 to 7000, further such as from 5500 to 6500 and even further such as from 5800 to 6200.

The copolymer comprises, for example, from 35 to 55 ethylene oxide and/or propylene oxide units and from 15 to 30 alkylene oxide units comprising from 6 to 40 carbon atoms.

In at least one embodiment, the copolymer is such that the ratio between the number of ethylene oxide and/or propylene oxide units and the number of alkylene oxide units comprising from 6 to 40 carbon atoms ranges from 1.5:1 to 2.5:1, such as from 1.8:1 to 2.3:1 and further such as from 1.9:1 to 2.1:1.

Such copolymers are described, for example, in document FR-A-2 425 848 and sold under the name “Elfacos® ST 9” by the company Akzo Nobel.

The pasty fatty substance may be present in the composition disclosed herein in an amount ranging, for example, from 1% to 25% by weight, such as from 1% to 18% by weight, and further such as from 1% to 10% by weight, relative to the total weight of the composition.

For example, the solid emulsion disclosed herein has a hardness after at least one month at 37° C. such that the penetration force is greater than 45 g, such as ranging from 45 g to 150 g.

The term “hardness after at least one month at 37° C.” means the hardness measured after storing the solid emulsion for at least one month at 37° C., for example, one month starting from the end of preparation of the solid emulsion.

For example, the solid emulsion has a hardness such that the penetration force after 24 hours at 20° C. (such as starting from the end of preparation of the solid emulsion) and after at least one month at 37° C. is greater than or equal to 45 grams (g), such as ranging from 45 g to 150 g.

In one embodiment, the hardness of the solid emulsion after at least one month at 37° C. ranges from 50 g to 130 g, such as from 60 g to 130 g and further such as from 70 g to 130 g.

The hardness of the emulsion is measured according to the following protocol:

At the end of preparation of the emulsion, it is poured while hot into a dish and is maintained at 20° C. for 24 hours. The penetration force of the solid emulsion is then measured using a texturometer sold under the name TA-XT2i by the company Rheo, equipped with a TA24 measuring spindle 4 mm in diameter, from the company Stevens, using the following measuring conditions:
trigger force=1.0 g,
pre-speed=2.0 mm/s,
penetration speed=0.5 mm/s,
penetration depth=2 mm.

The penetration force expressed in grams is read on the machine.

The solid emulsion is then stored for one month at 37° C., and the emulsion is then placed at 20° C. for 24 hours before measuring the hardness (termed the hardness after one month at 37° C.) under the same conditions described above.

The emulsion disclosed herein may comprise at least one filler.

The term “fillers” means colorless or white, mineral or synthetic particles of any shape, which are insoluble in the medium of the composition irrespective of the temperature at which the composition is manufactured.

The fillers may be mineral or organic and of any shape, in platelet form, spherical or oblong, irrespective of the crystallographic shape (for example lamellar, cubic, hexagonal, orthorhombic, etc.). Mention may be made, for example, of talc, mica, silica, kaolin, powders of polyamide (Nylon®), powders of poly-β-alanine, and powders of polyethylene, powders of tetrafluoroethylene polymers (Teflon®), lauroyllysine, starch, boron nitride, hollow polymer microspheres such as those of polyvinylidene chloride/acrylonitrile, for instance Expancel® (Nobel Industrie), acrylic acid copolymers, silicone resin microbeads (for example Tospearls® from Toshiba), polyorganosiloxane elastomer particles, precipitated calcium carbonate, magnesium carbonate and magnesium hydrocarbonate, hydroxyapatite, hollow silica microspheres, glass or ceramic microcapsules, and metal soaps derived from organic carboxylic acids comprising from 8 to 22 carbon atoms such as from 12 to 18 carbon atoms, for example zinc stearate, magnesium stearate, lithium stearate, zinc laurate or magnesium myristate.

The at least one filler may be present in the composition in an amount ranging, for example, from 0.1% to 25% by weight, such as from 1% to 20% by weight, and further such as from 5% to 15% by weight, relative to the total weight of the composition.

In one embodiment, the emulsion may comprise polymethyl methacrylate particles. For example, these particles are not film-forming, i.e., they do not form a continuous film when they are placed onto a support such as the skin.

The polymethyl methacrylate powders may be in the form of hollow or solid white spherical particles with a number-average size of micrometre order, ranging, for example, from 3 to 15 microns such as from 3 to 10 microns. The term “number-average size” means the size given by the statistical particle size distribution to half of the population, referred to as D50.

These polymethyl methacrylate particles may also be characterized by their density, which can vary, for example, as a function of the size of the spherical cavity of the particles.

As disclosed herein, the density of polymethyl methacrylate particles is assessed according to the following protocol, referred to as the packed density:
mass (m)=40 g of powder is poured into a measuring cylinder; the measuring cylinder is then placed on a Stav 2003 machine from Stampf Volumeter; the measuring cylinder is then subjected to 1500 packing motions; the final volume Vf of packed powder is then measured directly on the measuring cylinder. The packed density is determined by the ratio mass/Vf, in this instance 40/Vf (wherein Vf is expressed in cm³ and mass in g).

For example, the density of the polymethyl methacrylate particles that may be used herein may range from 0.3 to 1.5, such as from 0.5 to 1.5 and further such as from 1 to 1.5.

Among the polymethyl methacrylates that are suitable herein, mention may be made, for example, of the polymethyl methacrylate particles sold by the company Matsumoto Yushi Co. under the name “Micropearl M100”, by the company LCW under the name “Covabead LH 85” and those sold by the company Nihon Junyaku under the name “Jurymer MB1”.

The polymethyl methacrylate particles may be present in an amount ranging, for example, from 0.5% to 10% by weight, such as from 0.5% to 7% by weight, further such as from 1% to 20% by weight, and even further such as from 0.5% to 5% by weight, relative to the total weight of the composition.

The emulsion disclosed herein may comprise at least one dyestuff that may be chosen from water-soluble or liposoluble dyes, pigments and nacres, and mixtures thereof.

The term “pigments” as used herein means white or colored, mineral or organic particles, which are insoluble in the liquid organic phase, and which are intended to color and/or opacify the composition.

The term “nacres” as used herein means-iridescent particles, produced, for example, by certain molluscs in their shell or, alternatively, synthesized, which are insoluble in the medium of the composition.

The term “dyes” as used herein means compounds, such as organic compounds, which are soluble in fatty substances, for instance oils, or in an aqueous-alcoholic phase.

The pigments may be mineral or organic pigments. Pigments that may be used include, for example, metal oxides, for instance iron oxides (such as yellow, red, brown and black iron oxide), titanium dioxides, cerium oxide, zirconium oxide and chromium oxide; manganese violet, ultramarine blue, Prussian blue, cobalt blue, ferric blue, bismuth oxychloride, nacre, mica coated with titanium or with bismuth oxychloride, colored nacreous pigments such as titanium mica with iron oxides, titanium mica with, for example, ferric blue or chromium oxide, titanium mica with an organic pigment of the abovementioned type and nacreous pigments based on bismuth oxychloride, and mixtures thereof.

Iron oxide or titanium oxide pigments are used in at least one embodiment, for example.

The pigments may, for example, be treated with a hydrophobic agent to make them compatible with the organic phase of the composition. The hydrophobic-treatment agent may be chosen, for example, from silicones, such as methicones, dimethicones or perfluoroalkylsilanes; fatty acids, such as stearic acid; metal soaps, such as aluminium dimyristate, the aluminium salt of hydrogenated tallow glutamate, perfluoroalkyl phosphates, perfluoroalkylsilanes, perfluoroalkylsilazanes, polyhexafluoropropylene oxides, polyorganosiloxanes comprising perfluoroalkyl perfluoropolyether groups, and amino acids; N-acylamino acids or salts thereof; lecithin, isopropyl triisostearyl titanate, and mixtures thereof.

The N-acylamino acids may comprise an acyl group comprising from 8 to 22 carbon atoms, such as a 2-ethylhexanoyl, caproyl, lauroyl, myristoyl, palmitoyl, stearoyl or cocoyl group. The salts of these compounds may be, for example, aluminium, magnesium, calcium, zirconium, zinc, sodium or potassium salts. The amino acid may be, for example, lysine, glutamic acid or alanine.

The term “alkyl” mentioned in the compounds above means, for example, an alkyl group comprising from 1 to 30 carbon atoms such as from 5 to 16 carbon atoms.

Hydrophobic-treated pigments are described, for example, in European patent application EP-A-1 086 683.

The liposoluble dyes are chosen, for example, from Sudan Red, D&C Red No. 17, D&C Green No. 6, β-carotene, soybean oil, Sudan Brown, D&C Yellow No. 11, D&C Violet No. 2, D&C Orange No. 5, quinoline yellow, annatto and bromo acids.

The water-soluble dyes are chosen, for example, from beetroot juice, methylene blue and caramel.

The at least one dyestuff may be present in an amount ranging, for example, from 0.5% to 30% by weight, such as from 3% to 20% by weight, and further such as from 5% to 15% by weight, relative to the total weight of the composition.

In at least one embodiment, the emulsion may comprise at least one hydrophobic coated pulverulent material in an amount, for example, of less than or equal to 20% by weight relative to the total weight of the composition. The at least one hydrophobic coated pulverulent material may be chosen, for example, from pulverulent fillers and dyestuffs, such as the fillers and pigments as described above. The pulverulent material is coated with a hydrophobic agent as described above.

The composition may comprise other common cosmetic ingredients that may be chosen, for example, from hydrophilic or lipophilic gelling agents and thickeners, antioxidants, fragrances, preserving agents, neutralizers, sunscreens, vitamins, moisturizers, self-tanning compounds, anti-wrinkle active agents, emollients, hydrophilic or lipophilic active agents, free-radical scavengers, sequestering agents and film-forming agents, and mixtures thereof.

A person skilled in the art will take care to select this or these optional compound(s), and/or the amount thereof, such that the advantageous properties of the composition disclosed herein are not, or are not substantially, adversely affected by the envisaged addition.

The composition disclosed herein may be prepared, for example, according to the following procedure:

The mixture of the constituents of the oily phase is first prepared by mixing together and heating, at a temperature ranging from 70° C. to 120° C., the waxes, the block copolymer of ethylene oxide and/or of propylene oxide and of an alkylene oxide comprising from 6 to 40 carbon atoms, and the non-volatile oils, followed by adding with stirring, at a temperature ranging from 60° C. to 80° C., the volatile oils, the fillers and the pigments.

The mixture of the constituents of the aqueous phase, comprising the water, the surfactants and the water-miscible solvents is prepared separately, with heating to a temperature ranging from 60° C. to 80° C.

Next, the aqueous phase is added to the oily phase, at a temperature ranging from 60° C. to 80° C. and the resulting mixture is stirred using a turbomixer until the water-in-oil emulsion is obtained. The emulsion is then poured into a container, for example, a dish, and then cooled to room temperature until the solid emulsion is obtained.

The disclosure is illustrated in greater detail by the non-limiting examples described below.

Other than in the examples, or where otherwise indicated, all numbers expressing quantities of ingredients, reaction conditions, and so forth used in the specification and claims are to be understood as being modified in all instance by the term “about.” Accordingly, unless indicated to the contrary, the numerical parameters set forth in the following specification and attached claims are approximations that may vary depending upon desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific examples are reported as precisely as possible. Any numerical value, however, inherently contain certain errors necessarily resulting from the standard deviation found in their respective testing measurements.

### EXAMPLE 1

A solid foundation having the composition below was prepared:

| Oily phase: | | |
|---|---|---|
| Candelilla wax | 1.8 | g |
| Ozokerite | 1.2 | g |
| Isotridecyl isononanoate | 9.0 | g |
| Cyclopentasiloxane | 22.5 | g |
| Iron oxides coated with disodium stearoyl glutamate and | 2.2 | g |
| aluminium hydroxide | | |
| Titanium dioxide coated with disodium stearoyl glutamate and | 7.8 | g |
| aluminium hydroxide | | |
| Hydrophobic fumed silica (Aerosil R972 from Degussa) | 0.5 | g |
| Silica microbeads | 9.0 | g |
| Polymethyl methacrylate | 3 | g |
| Titanium dioxide nanopigments | 3 | g |
| Copolymer of ethylene oxide (45 EO) and epoxydodecane | 4 | g |
| (22 mol) sold under the name “Elfacos ® ST 9” by the | | |
| company Akzo Nobel | | |
| Polyglyceryl-4 isostearate | 1.50 | g |
| Silicone comprising at least one polyglycerolated chain - | 2 | g |
| (KF 6104 from Shin-Etsu) | | |
| Aqueous phase: | | |
| Water | 20 | g |
| Butylene glycol | 3 | g |
| Glycerol | 5 | g |
| Magnesium sulphate | 1 | g |
| Mixture of oxyethylenated oxypropylenated (18 EO/18 PO) | 2 | g |
| polydimethylsiloxane, cyclopentasiloxane and water (10/88/2) | | |
| (DC 2-5225 C from Dow Corning) | | |
| Preserving agents | 1.50 | g |

The composition was prepared according to the following procedure:

The mixture comprising the waxes, the copolymer Elfacos ST 9 and the non-volatile oils was heated to 90° C. until a clear, uniform mixture was obtained; the volatile oils, the pigments and the fillers were then added with stirring, at 70° C. The mixture of the ingredients of the aqueous phase was then prepared with heating to 70° C., and the aqueous phase was then introduced at 70° C. into the oily phase, with stirring until the water-in-oil emulsion was obtained.

The emulsion was poured at 70° C. into a preheated dish and was then allowed to cool to room temperature until the solid emulsion was obtained.

A solid foundation was obtained, having a hardness of 118.2 g measured at 24 hours at 20° C. and a hardness of greater than 45 g measured after storage at 37° C. for one month, the hardness measurements being performed according to the protocol described above.

When applied to the skin, the foundation was rich and creamy.

### EXAMPLE 2

A solid foundation having the composition below was prepared:

| Oily phase: | | |
|---|---|---|
| Candelilla wax | 1.8 | g |
| Ozokerite | 1.2 | g |
| Isotridecyl isononanoate | 9.0 | g |
| Cyclopentasiloxane | 22.5 | g |
| Iron oxides coated with disodium stearoyl glutamate and | 2.2 | g |
| aluminium hydroxide | | |
| Titanium dioxide coated with disodium stearoyl glutamate and | 7.8 | g |
| aluminium hydroxide | | |
| Hydrophobic fumed silica (Aerosil R972 from Degussa) | 0.5 | g |
| Silica microbeads | 9.0 | g |
| Polymethyl methacrylate | 3 | g |
| Titanium dioxide nanopigments | 3 | g |
| Copolymer of ethylene oxide (45 EO) and epoxydodecane | 4 | g |
| (22 mol) sold under the name “Elfacos ® ST 9” by | | |
| the company Akzo Nobel | | |
| Polyglyceryl-4 isostearate | 1.50 | g |
| Aqueous phase: | | |
| Water | 20 | g |
| Butylene glycol | 3 | g |
| Glycerol | 5 | g |
| Magnesium sulfate | 1 | g |
| Cetyl dimethicone copolyol (Abil ® EM 90 from the | 2 | g |
| company Goldschmidt) | | |
| Mixture of oxyethylenated oxypropylenated (18 EO/18 PO) | 2 | g |
| polydimethylsiloxane, cyclopentasiloxane and water (10/88/2) | | |
| (DC 2-5225 C from Dow Corning) | | |
| Preserving agents | 1.50 | g |

The composition was prepared according to the following procedure:

The mixture comprising the waxes, the copolymer Elfacos ST 9 and the non-volatile oils was heated to 90° C. until a clear, uniform mixture was obtained; the volatile oils, the pigments and the fillers were then added with stirring, at 70° C. The mixture of the ingredients of the aqueous phase was then prepared with heating to 70° C., and the aqueous phase was then introduced at 70° C. into the oily phase, with stirring until the water-in-oil emulsion was obtained.

The emulsion was poured at 70° C. into a preheated dish and was then allowed to cool to room temperature until the solid emulsion was obtained.

A solid foundation was obtained, having a hardness of 98 g measured at 24 hours at 20° C. and a hardness of 87 g measured after storage at 37° C. for one month, the hardness measurements being performed according to the protocol described above. This solid foundation thus had good hardness stability after storage for one month.

When applied to the skin, the foundation was rich and creamy.

### EXAMPLE 3

A solid foundation having the composition below was prepared:

| Oily phase: | | |
|---|---|---|
| Candelilla wax | 1.8 | g |
| Ozokerite | 1.2 | g |
| Isodecyl neopentanoate | 10.2 | g |
| Cyclopentasiloxane | 22.5 | g |
| Iron oxides coated with disodium stearoyl glutamate and | 2.4 | g |
| aluminium hydroxide | | |
| Titanium dioxide coated with disodium stearoyl glutamate and | 7.6 | g |
| aluminium hydroxide | | |
| Hydrophobic fumed silica (Aerosil R972 from Degussa) | 0.5 | g |
| Silica microbeads | 9.0 | g |
| Polymethyl methacrylate | 3 | g |
| Titanium dioxide nanopigments | 3 | g |
| Copolymer of ethylene oxide (45 EO) and epoxydodecane | 4 | g |
| (22 mol) sold under the name “Elfacos ® ST 9” by | | |
| the company Akzo Nobel | | |
| Polyglyceryl-3 diisostearate | 0.3 | g |
| Aqueous phase: | | |
| Water | 20 | g |
| Butylene glycol | 3 | g |
| Glycerol | 5 | g |
| Magnesium sulfate | 1 | g |
| Cetyl dimethicone copolyol (Abil ® EM 90 from the | 2 | g |
| company Goldschmidt) | | |
| Mixture of oxyethylenated oxypropylenated (18 EO/18 PO) | 2 | g |
| polydimethylsiloxane, cyclopentasiloxane and water (10/88/2) | | |
| (DC 2-5225 C from Dow Corning) | | |
| Preserving agents | 1.50 | g |

The emulsion was prepared in the same manner as that of Example 1.

A solid foundation was obtained, having a hardness of 87 g measured at 24 hours at 20° C. and a hardness of 78 g measured after storage at 37° C. for one month, the hardness measurements being performed according to the protocol described above. This solid foundation thus had good hardness stability after storage for one month.

When applied to the skin, the foundation was rich and creamy.

What is claimed is:

1. A solid water-in-oil emulsion comprising an aqueous phase emulsified in a fatty phase comprising at least one ester oil and at least one wax, and at least one silicone surfactant comprising at least one polyhydroxylated chain.

2. The emulsion according to claim 1, wherein the emulsion has a hardness after at least one month at 37° C. such that the penetration force is greater than or equal to 45 g.

3. A solid water-in-oil emulsion comprising an aqueous phase emulsified with at least one emulsifying surfactant in a fatty phase comprising at least one ester oil and at least one wax, and at least one alkyl dimethicone copolyol chosen from C₈-C₂₂ alkyl dimethicone copolyols, wherein the emulsion has, after at least one month at 37° C., a hardness such that the penetration force is greater than or equal to 45 g.

4. The emulsion according to claim 2, wherein the hardness after at least one month at 37° C. is such that the penetration force ranges from 45 g to 150 g.

5. The emulsion according to claim 4, wherein the hardness after at least one month at 37° C. is such that the penetration force ranges from 70 g to 130 g.

6. The emulsion according to claim 1, wherein the at least one ester oil is chosen from monoesters.

7. The emulsion according to claim 6, wherein the at least one ester oil is chosen from monoesters of formula R₁COOR₂ wherein R₁ is chosen from linear and branched hydrocarbon-based chains comprising from 4 to 40 carbon atoms, and R₂ is chosen from branched hydrocarbon-based chains comprising from 3 to 40 carbon atoms.

8. The emulsion according to claim 7, wherein R₁ is chosen from linear and branched hydrocarbon-based chains comprising from 7 to 20 carbon atoms and/or R₂ is chosen from branched hydrocarbon-based chains comprising from 16 to 26 carbon atoms.

9. The emulsion according to claim 1, wherein the at least one ester oil is chosen from isodecyl neopentanoate, isocetyl octanoate, isononyl isononanoate, isodecyl isononanoate, tridecyl isononanoate, hexyl laurate, 2-hexyldecyl laurate, isopropyl myristate, isocetyl myristate, isotridecyl myristate, 2-octyldodecyl myristate, isopropyl palmitate, 2-ethylhexyl palmitate, isooctyl palmitate, isocetyl palmitate, isodecyl palmitate, isostearyl palmitate, 2-octyldecyl palmitate, isopropyl isostearate, 2-octyldodecyl stearate, isostearyl isostearate and 2-octyldodecyl erucate, and mixtures thereof.

10. The emulsion according to claim 1, wherein the at least one ester oil is present in an amount ranging from 5% to 20% by weight relative to the total weight of the emulsion.

11. The emulsion according to claim 10, wherein the at least one ester oil is present in an amount ranging from 8% to 12% by weight relative to the total weight of the emulsion.

12. The emulsion according to claim 1, wherein the at least one wax has a hardness ranging from 5 MPa to 9 MPa.

13. The emulsion according to claim 12, wherein the at least one wax has a hardness ranging from 7 MPa to 9 MPa.

14. The emulsion according to claim 1, wherein the at least one wax is chosen from carnauba wax, microcrystalline waxes, ozokerites, hydrogenated jojoba oil, polyethylene waxes, poly(C₂₄-C₂₈)alkyl methyl dimethyl siloxane waxes, palm butter, C₂₀-C₄₀ alkyl stearate, stearyl benzoate and shellac wax.

15. The emulsion according to claim 14, wherein the at least one wax is chosen from carnauba wax, ozokerites, hydrogenated jojoba oil and polyethylene waxes.

16. The emulsion according to claim 1, wherein the at least one wax is chosen from candelilla wax and ozokerite.

17. The emulsion according to claim 1, wherein the at least one wax is present in an amount ranging from 1% to 10% by weight relative to the total weight of the emulsion.

18. The emulsion according to claim 17, wherein the at least one wax is present in an amount ranging from 2% to 5% by weight relative to the total weight of the emulsion.

19. The emulsion according to claim 3, wherein the hardness after at least one month at 37° C. is such that the penetration force ranges from 45 g to 150 g.

20. The emulsion according to claim 19, wherein the hardness after at least one month at 37° C. is such that the penetration force ranges from 70 g to 130 g.

21. The emulsion according to claim 3, wherein the at least one ester oil is chosen from monoesters.

22. The emulsion according to claim 21, wherein the at least one ester oil is chosen from monoesters of formula R₁COOR₂ wherein R₁ is chosen from linear and branched hydrocarbon-based chains comprising from 4 to 40 carbon atoms, and R₂ is chosen from branched hydrocarbon-based chains comprising from 3 to 40 carbon atoms.

23. The emulsion according to claim 22, wherein R₁ is chosen from linear and branched hydrocarbon-based chains comprising from 7 to 20 carbon atoms and/or R₂ is chosen from branched hydrocarbon-based chains comprising from 16 to 26 carbon atoms.

24. The emulsion according to claim 3, wherein the at least one ester oil is chosen from isodecyl neopentanoate, isocetyl octanoate, isononyl isononanoate, isodecyl isononanoate, tridecyl isononanoate, hexyl laurate, 2-hexyldecyl laurate, isopropyl myristate, isocetyl myristate, isotridecyl myristate, 2-octyldodecyl myristate, isopropyl palmitate, 2-ethylhexyl palmitate, isooctyl palmitate, isocetyl palmitate, isodecyl palmitate, isostearyl palmitate, 2-octyldecyl palmitate, isopropyl isostearate, 2-octyldodecyl stearate, isostearyl isostearate and 2-octyldodecyl erucate.

25. The emulsion according to claim 3, wherein the at least one ester oil is present in an amount ranging from 5% to 20% by weight relative to the total weight of the emulsion.

26. The emulsion according to claim 25, wherein the at least one ester oil is present in an amount ranging from 8% to 12% by weight relative to the total weight of the emulsion.

27. The emulsion according to claim 3, wherein the at least one wax has a hardness ranging from 5 MPa to 9 MPa.

28. The emulsion according to claim 27, wherein the at least one wax has a hardness ranging from 7 MPa to 9 MPa.

29. The emulsion according to claim 3, wherein the at least one wax is chosen from carnauba wax, microcrystalline waxes, ozokerites, hydrogenated jojoba oil, polyethylene waxes, poly(C₂₄-C₂₈)alkyl methyl dimethyl siloxane waxes, palm butter, C₂₀-C₄₀ alkyl stearate, stearyl benzoate and shellac wax.

30. The emulsion according to claim 3, wherein the at least one wax is chosen from candelilla wax and ozokerite.

31. The emulsion according to claim 3, wherein the at least one wax is present in an amount ranging from 1% to 10% by weight relative to the total weight of the emulsion.

32. The emulsion according to claim 31, wherein the at least one wax is present in an amount ranging from 2% to 5% by weight relative to the total weight of the emulsion.

33. The emulsion according to claim 1, wherein the at least one silicone surfactant comprising at least one polyhydroxylated chain is chosen from silicone polymers of formula (I′) below:
R¹ₐR²_{b}R³_{c}SiO_{(4-a-b-c)/2} (I′)
wherein:
a) a ranges from 1 to 2.5; and b and c, which may be identical or different, range from 0.001 to 1.5,
b) R¹, which may be identical or different, is chosen from:
C₁ to C₃₀ alkyl radicals, optionally substituted with at least one entity chosen from fluorine atoms and amino and carboxyl groups,
aryl and aralkyl radicals, and
radicals of formula (II):
—C_{d}H_{2d}—O—(C₂H₄O)ₑ(C₃H₆O)_{f}R⁴ (II)
wherein:
R⁴ is chosen from C₄ to C₃₀ hydrocarbon-based radicals and a radical R⁵—(CO)— wherein R⁵ is chosen from C₁ to C₃₀ hydrocarbon-based radicals, and
d, e and f are integers such that d ranges from 0 to 15, and e and f, which may be identical or different, range from 0 to 50, and
combinations thereof,
c) R² is a radical of formula (III) below:
-Q-O—X (III)
wherein:
Q is a divalent radical chosen from divalent C₂ to C₂₀ hydrocarbon-based radical which may comprise at least one bond chosen from ether bonds and ester bonds, and
X is a polyhydroxylated hydrocarbon-based radical,
d) R³ is an organosiloxane group of formula (IV): 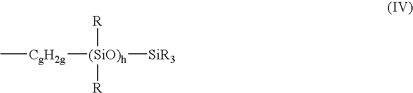
wherein:
radicals R, which may be identical or different, are each a radical chosen from C₁ to C₃₀ alkyl radicals, optionally substituted with at least one fluorine atom, and aryl and aralkyl radicals,
g and h are integers such that g ranges from 1 to 5 and h ranges from 0 to 500.

34. The emulsion according to claim 33, wherein the silicone polymers are chosen from polymers of the formula (I′) wherein R¹ is a radical chosen from C₁ to C₁₀ alkyl radicals.

35. The emulsion according to claim 34, wherein R¹ is a radical chosen from C₁ to C₄ alkyl radicals.

36. The emulsion according to claim 33, wherein the silicone polymers are chosen from polymers of the formula (I′) wherein:
a ranges from 1 to 1.4,
b and c, which may be identical or different, range from 0.02 to 0.04,
R¹ is a radical chosen from C₁ to C₁₀ alkyl radicals,
R² is a radical of formula (IIIA):
—C₃H₆O[CH₂CH(OH)CH₂O]ₙH (IIIA)
wherein n ranges from 1 to 5, and
R³ is a radical of formula (IVA):
—C₂H₄(CH₃)₂SiO[(CH₃)₂SiO]ₘSi(CH₃)₃ (IVA)
wherein m ranges from 3 to 9.

37. The emulsion according to claim 36, wherein the silicone polymers are chosen from polymers of the formula (I′) wherein:
a ranges from 1 to 1.4,
b and c, which may be identical or different, range from 0.02 to 0.04, and
R¹ is a methyl radical,
R² is a radical of the formula (IIIA) wherein n ranges from 1 to 5, and
R³ is a radical of the formula (IVA) wherein m ranges from 3 to 9.

38. The emulsion according to claim 1, wherein the at least one silicone surfactant comprising at least one polyhydroxylated chain is chosen from emulsifying elastomeric crosslinked organopolysiloxanes comprising at least one polyhydroxylated chain.

39. The emulsion according to claim 38, wherein the at least one polyhydroxylated chain is chosen from polyglycerolated chains.

40. The emulsion according to claim 38, wherein the elastomeric crosslinked organopolysiloxanes are obtained by crosslinking addition reaction of diorganopolysiloxane comprising at least one hydrogen linked to silicon and polyhydroxylated compounds comprising at least one ethylenically unsaturated group.

41. The emulsion according to claim 40, wherein the crosslinking addition reaction is carried out in the presence of a platinum catalyst.

42. The emulsion according to claim 38, wherein the elastomeric crosslinked organopolysiloxanes are obtained by crosslinking addition reaction of (A) diorganopolysiloxane comprising at least two hydrogens each linked to a silicon, and (B) glycerolated compounds comprising at least two ethylenically unsaturated groups.

43. The emulsion according to claim 42, wherein the crosslinking addition reaction is carried out in the presence of (C) a platinum catalyst.

44. The emulsion according to claim 42, wherein the compound (B) is chosen from polyglycerolated compounds of formula (B′) below:
CₘH₂ₘ₋₁—O—[Gly]n-CₘH₂ₘ₋₁ (B′)
wherein:
m is an integer ranging from 2 to 6,
n is an integer ranging from 2 to 200, and
Gly is chosen from —CH₂—CH(OH)—CH₂—O— and —CH₂—CH(CH₂OH)—O—.

45. The emulsion according to claim 44, wherein in formula (B′), n ranges from 2 to 5.

46. The emulsion according to claim 1, wherein the at least one silicone surfactant comprising at least one polyhydroxylated chain is present in an amount ranging from 0.1% to 5% by weight relative to the total weight of the emulsion.

47. The emulsion according to claim 46, wherein the at least one silicone surfactant comprising at least one polyhydroxylated chain is present in an amount ranging from 1% to 3% by weight relative to the total weight of the emulsion.

48. The emulsion according to claim 3, wherein the C₈-C₂₂ alkyl dimethicone copolyols are chosen from compounds of formula (I) below: 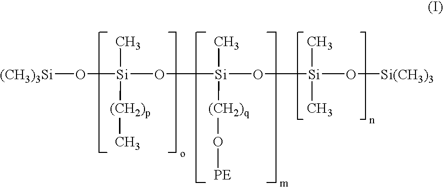
wherein:
PE is a radical (—C₂H₄O)ₓ—(C₃H₆O)_{y}—R, wherein R is chosen from a hydrogen atom and alkyl radicals comprising from 1 to 4 carbon atoms, x ranges from 0 to 100, and y ranges from 0 to 80, wherein x and y are not simultaneously 0,
m ranges from 1 to 40,
n ranges from 10 to 200,
o ranges from 1 to 100,
p ranges from 7 to 21, and
q ranges from 0 to 4.

49. The emulsion according to claim 3, wherein the at least one alkyl dimethicone copolyol chosen from C₈-C₂₂ alkyl dimethicone copolyols is present in an amount ranging from 0.1% to 5% by weight relative to the total weight of the emulsion.

50. The emulsion according to claim 49, wherein the at least one alkyl dimethicone copolyol chosen from C₈-C₂₂ alkyl dimethicone copolyols is present in an amount ranging from 1% to 3% by weight relative to the total weight of the emulsion.

51. The emulsion according to claim 1, further comprising at least one additional surfactant chosen from fatty acid esters of polyols, alkyl and alkoxy dimethicone copolyols comprising at least one chain chosen from alkyl and alkoxy chains that is pendent or at the end of the silicone skeleton, and polymers chosen from polyoxyalkylenated fatty acid esters of glycol.

52. The emulsion according to claim 51, wherein the at least one additional surfactant is chosen from C₈-C₂₂ alkyl dimethicone copolyols of formula (I) below: 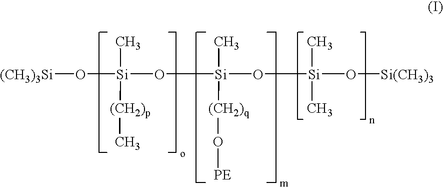
wherein:
PE is a radical (—C₂H₄O)ₓ—(C₃H₆O)_{y}—R, wherein R is chosen from a hydrogen atom and alkyl radicals comprising from 1 to 4 carbon atoms, x ranges from 0 to 100, and y ranges from 0 to 80, wherein x and y are not simultaneously 0,
m ranges from 1 to 40,
n ranges from 10 to 200,
o ranges from 1 to 100,
p ranges from 7 to 21, and
q ranges from 0 to 4.

53. The emulsion according to claim 3, further comprising at least one additional surfactant chosen from fatty acid esters of polyols, and polymers chosen from polyoxyalkylenated fatty acid esters of glycol, polyhydroxylated silicones, emulsifying silicone elastomers, and silicone surfactants comprising at least one polyhydroxylated chain.

54. The emulsion according to claim 51, wherein the at least one additional surfactant is present in an amount ranging from 0.05% to 3% by weight relative to the total weight of the emulsion.

55. The emulsion according to claim 54, wherein the at least one additional surfactant is present in an amount ranging from 0.1% to 2% by weight relative to the total weight of the emulsion.

56. The emulsion according to claim 1, wherein the fatty phase further comprises at least one volatile silicone oil.

57. The emulsion according to claim 56, wherein the at least one volatile silicone oil is chosen from octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane.

58. The emulsion according to claim 56, wherein the at least one volatile silicone oil is present in an amount ranging from 5% to 40% by weight relative to the total weight of the emulsion.

59. The emulsion according to claim 58, wherein the at least one volatile silicone oil is present in an amount ranging from 15% to 30% by weight relative to the total weight of the emulsion.

60. The emulsion according to claim 56, further comprising at least one additional oil other than the at least one ester oil and the at least one volatile silicone oil.

61. The emulsion according to claim 60, wherein the at least one additional oil is chosen from volatile and non-volatile hydrocarbon-based oils and fluoro oils.

62. The emulsion according to claim 60, wherein the at least one additional oil is present in an amount ranging from 0.1% to 10% by weight relative to the total weight of the emulsion.

63. The emulsion according to claim 62, wherein the at least one additional oil is present in an amount ranging from 0.1% to 5% by weight relative to the total weight of the emulsion.

64. The emulsion according to claim 1, further comprising water in an amount ranging from 10% to 40% by weight relative to the total weight of the emulsion.

65. The emulsion according to claim 64, further comprising water in an amount ranging from 15% to 25% by weight relative to the total weight of the emulsion.

66. The emulsion according to claim 1, further comprising at least one water-miscible solvent chosen from monoalcohols comprising from 2 to 6 carbon atoms, polyols comprising from 2 to 20 carbon atoms, and glycol ethers comprising from 3 to 16 carbon atoms.

67. The emulsion according to claim 66, wherein the at least one water-miscible solvent is chosen from glycerol, propylene glycol, butylene glycol, pentylene glycol, hexylene glycol, dipropylene glycol, diethylene glycol, mono-, di- and tripropylene glycol (C₁-C₄)alkyl ethers, mono-, di- and triethylene glycol (C₁-C₄)alkyl ethers, ethanol and isopropanol.

68. The emulsion according to claim 66, wherein the at least one water-miscible organic solvent is present in an amount ranging from 1% to 20% by weight relative to the total weight of the emulsion.

69. The emulsion according to claim 68, wherein the at least one water-miscible organic solvent is present in an amount ranging from 3% to 15% by weight relative to the total weight of the emulsion.

70. The emulsion according to claim 1, further comprising at least one pasty fatty substance.

71. The emulsion according to claim 70, wherein the at least one pasty fatty substance is chosen from block copolymers of ethylene oxide and/or propylene oxide and of an alkylene oxide comprising from 6 to 40 carbon atoms, wherein the copolymers have a weight-average molecular weight ranging from 5000 to 8000.

72. The emulsion according to claim 1, further comprising at least one filler.

73. The emulsion according to claim 72, wherein the at least one filler is chosen from talc, mica, silica, kaolin, polyamide powders, poly-β-alanine powders, polyethylene powders, tetrafluoroethylene polymer powders, lauroyllysine, starch, boron nitride, hollow microspheres of polyvinylidene chloride/acrylonitrile, hollow microspheres of acrylic acid copolymers, silicone resin microbeads, polyorganosiloxane elastomer particles, precipitated calcium carbonate, magnesium carbonate and magnesium hydrocarbonate, hydroxyapatite, hollow silica microspheres, glass or ceramic microcapsules, and metal soaps derived from organic carboxylic acids comprising from 8 to 22 carbon atoms.

74. The emulsion according to claim 72, wherein the at least one filler is present in an amount-ranging from 0.1% to 25% by weight relative to the total weight of the emulsion.

75. The emulsion according to claim 74, wherein the at least one filler is present in an amount ranging from 5% to 15% by weight relative to the total weight of the emulsion.

76. The emulsion according to claim 1, further comprising polymethyl methacrylate particles.

77. The emulsion according to claim 76, wherein the polymethyl methacrylate particles are present in an amount ranging from 0.5% to 10% by weight relative to the total weight of the emulsion.

78. The emulsion according to claim 77, wherein the polymethyl methacrylate particles are present in an amount ranging from 0.5% to 5% by weight relative to the total weight of the emulsion.

79. The emulsion according to claim 1, further comprising at least one dyestuff.

80. The emulsion according to claim 1, further comprising at least one hydrophobic coated pulverulent material.

81. The emulsion according to claim 80, wherein the at least one hydrophobic coated pulverulent material is chosen from hydrophobic coated pulverulent fillers and dyestuffs.

82. The emulsion according to claim 80, wherein the at least one hydrophobic coated pulverulent material is coated with at least one hydrophobic agent chosen from silicones, fatty acids, metal soaps, perfluoroalkyl phosphates, perfluoroalkyl silanes, perfluoroalkyl silazanes, polyhexafluoropropylene oxides, polyorganosiloxanes comprising perfluoroalkyl perfluoropolyether groups, amino acids, N-acylamino acids or salts thereof, lecithin and isopropyl triisostearyl titanate.

83. The emulsion according to claim 80, wherein the at least one hydrophobic coated pulverulent material is present in an amount of less than or equal to 20% by weight relative to the total weight of the emulsion.

84. The emulsion according to claim 1, further comprising at least one cosmetic ingredient chosen from hydrophilic and lipophilic gelling agents and thickeners, antioxidants, fragrances, preserving agents, neutralizers, sunscreens, vitamins, moisturizers, self-tanning compounds, anti-wrinkle active agents, emollients, hydrophilic and lipophilic active agents, free-radical scavengers, sequestering agents and film-forming agents.

85. The emulsion according to claim 1, wherein the emulsion is in a form chosen from a foundation, an eyeshadow, a makeup rouge, a concealer product, a body makeup product, a lipstick, a skincare base, a care cream, a lipcare composition, an antisun composition, a self-tanning composition, and a deodorant.

86. The emulsion according to claim 3, wherein the emulsion is in a form chosen from a foundation, an eyeshadow, a makeup rouge, a concealer product, a body makeup product, a lipstick, a skincare base, a care cream, a lipcare composition, an antisun composition, a self-tanning composition, and a deodorant.

87. A non-therapeutic cosmetic process for making up or caring for skin, comprising applying to the skin a solid water-in-oil emulsion comprising an aqueous phase emulsified in a fatty phase comprising at least one ester oil and at least one wax, and at least one silicone surfactant comprising at least one polyhydroxylated chain.

88. A non-therapeutic cosmetic process for making up or caring for skin, comprising applying to the skin a solid water-in-oil emulsion comprising an aqueous phase emulsified with at least one emulsifying surfactant in a fatty phase comprising at least one ester oil and at least one wax, and at least one alkyl dimethicone copolyol chosen from C₈-C₂₂ alkyl dimethicone copolyols, wherein the emulsion has, after at least one month at 37° C., a hardness such that the penetration force is greater than or equal to 45 g.
